(19) United States
(12) Patent Application Publication
(10) Pub. No.: US 2023/0287473 A1
(43) Pub. Date: Sep. 14, 2023

(54) BACTERIAL SPORE GERMINATION ASSAY OF MICROBIOTA DISRUPTION

(71) Applicant: University of Virginia Patent Foundation, Charlottesville, VA (US)

(72) Inventors: Nathan Swami, Charlottesville, VA (US); Cirle Alcantrara Warren, Waynesboro, VA (US); John Hudson Moore, II, Waynesboro, VA (US); Carlos Honrado, Macedo Cavaleiros (PT); Armita Salahi, Charlottesville, VA (US)

(21) Appl. No.: 18/006,236

(22) PCT Filed: Jul. 21, 2021
(86) PCT No.: PCT/US2021/070922

§ 371 Date: Jan. 20, 2023

Related U.S. Application Data
(60) Provisional application No. 63/054,601, filed on Jul. 21, 2020.

(51) Int. Cl. C12Q 1/04; G01N 15/10

(52) U.S. Cl. CPC C12Q 1/04; G01N 15/1031; G01N 2015/1006

(57) ABSTRACT

A method for quantifying bacterial spore germination can include creating an ex vivo assay including bacteria spores and a homogenized stool sample. The ex vivo assay can be loaded into a microfluidic chip. Vegetative bacteria and the bacteria spores can be detected by sampling the mixture in the microfluidic chip using impedance cytometry to assess disruption of host microbiota.

### CLAIM OF PRIORITY

This patent application claims the benefit of priority, under 35 U.S.C. § 119(e), to Swami, Nathan, U.S. Patent Application Ser. No. 63/054,601, entitled “SYSTEM AND METHOD FOR QUANTIFYING BACTERIAL SPORE GERMINATION BY SINGLECELL IMPEDANCE CYTOMETRY FOR ASSESSMENT OF HOST MICROBIOTA SUSCEPTIBILITY TO INFECTION,” filed on Jul. 21, 2020 (Attorney Docket No. 1036.312PRV), which is hereby incorporated by reference herein in its entirety.

### STATEMENT REGARDING FEDERALLY SPONSORED RESEARCH OR DEVELOPMENT

This invention was made with government support under award number AI130902 awarded by the National Institutes of Health, and under award number FA2386-18-1-4100 awarded by the Air Force Office of Scientific Research. The government has certain rights in this invention.

### BACKGROUND

Infections are common in the human body and can be caused by a variety of parasites, such as bacteria. Intestinal infections occur in the intestines of humans (and other animals) and are commonly caused by a disruption of the gut microbiota microbiota, which normally operates under a delicate balance of bacterial diversity. The balance of the microbiota can become disrupted due to aging or due to ingestion of exogenous materials, such as antibiotic drugs for treatment of infections in various parts of the body. Once the microbiota becomes compromised or imbalanced, bacteria (which may already present in the intestine or can be introduced exogenously) can cause a bacterial infection of the intestine. Due to the prevalence of this issue, tests can be used to determine the probability that an antibiotic will create an environment prone to infection in a human intestine.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a schematic view of bacteria.

FIG. 1B illustrates a schematic view of a timeline.

FIG. 1C illustrates a schematic view of a timeline.

FIG. 1D illustrates a schematic view of a process.

FIG. 2A-2C illustrate graphs.

FIG. 2B illustrates a graph.

FIG. 2C illustrates a graph.

FIG. 3A illustrates a schematic view of a vegetative bacteria.

FIG. 3B illustrates a schematic view of a bacteria spore.

FIG. 3C illustrates a graph.

FIG. 4A-4F illustrate graphs.

FIG. 5A-5J illustrate graphs.

FIGS. 6A-6C illustrate graphs.

FIG. 7A illustrates a schematic view of bacteria.

FIG. 7B illustrates a graph.

FIG. 8 illustrates a schematic view of a system.

FIG. 9A-9C illustrate graphs.

FIG. 10A-10B illustrate graphs.

FIG. 11A-11B illustrate graphs.

FIG. 12 illustrates a schematic view of a process.

FIG. 13 illustrates a schematic view of a process.

FIG. 14 illustrates a schematic view of a process.

### DETAILED DESCRIPTION

Many gastro-intestinal infections, such as a Clostridioides difficile (C. difficile) infection, require germination of ingested spores for enabling bacterial outgrowth and host colonization. Spore germination rate in the colon depends on microbiota composition and its disruption by antibiotic treatment since secretions by commensal bacteria can modulate primary to secondary bile salt levels to control germination. Assessment of C. difficile spore germination typically requires measurement of colony-forming units, which is labor intensive and takes at least 24 hours to perform but is regularly required due to the high recurrence rates of nosocomial antibiotic-associated diarrhea.

This application discusses devices and methods that can help address these issues by using a rapid method to assess spore germination by using high throughput single-cell impedance cytometry (for example, at 300 events per second) to quantify live bacterial cells, by gating for their characteristic electrophysiology versus spores, so that germination can be assessed after four hours of culture based on a detection limit of about 100 live cells. To detect the phenotype of live C. difficile bacteria, their characteristically higher cytoplasmic conductivity can be used versus that of spore aggregates and non-viable C. difficile forms, which causes a distinctive high-frequency (e.g., 10 MHz) impedance phase dispersion within moderately conductive media (0.8-1.6 S/m). In this manner, significant differences in spore germination rates can be detected within just 4 hours, as validated with increasing primary bile salt levels in vitro and using ex vivo microbiota samples from an antibiotic-treated mouse model to assess susceptibility to C. difficile infection. This method can be used as a rapid diagnostic tool for assessing host microbiota susceptibility to bacterial colonization after key antibiotic treatments.

An aspect of an embodiment of the present invention provides a system, method and computer readable medium for, among other things, quantifying bacterial spore germination by single-cell impedance cytometry for assessment of host microbiota susceptibility to Clostridioides difficile infection.

An aspect of an embodiment of the present invention provides a system, method and computer readable medium for, among other things, quantifying Clostridioides difficile spore germination based on bacterial electrophysiology for assessing microbiota susceptibility to infection.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

The above discussion is intended to provide an overview of subject matter of the present patent application. It is not intended to provide an exclusive or exhaustive explanation of the invention. The description below is included to provide further information about the present patent application.

### 1. Introduction

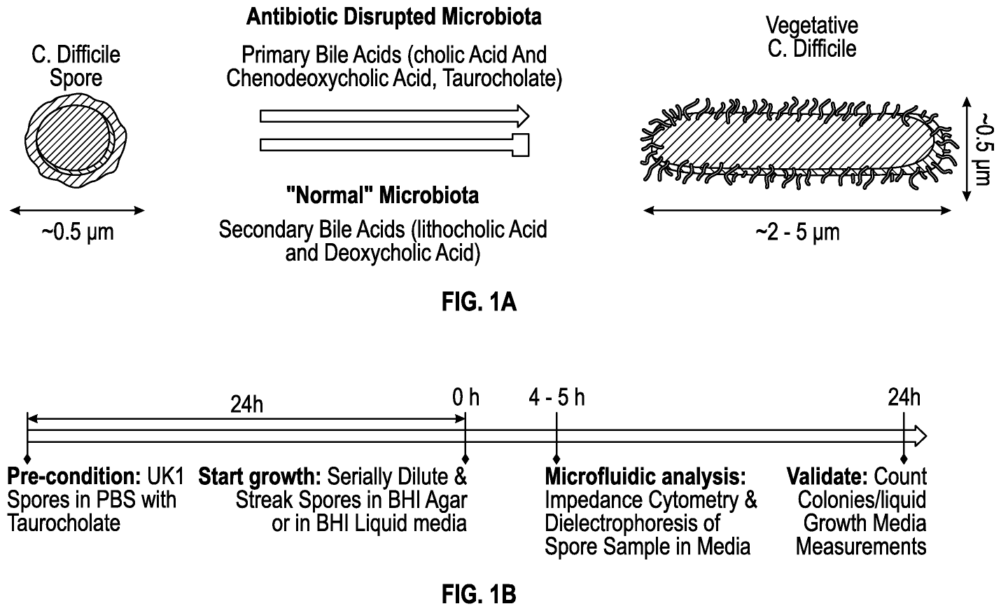

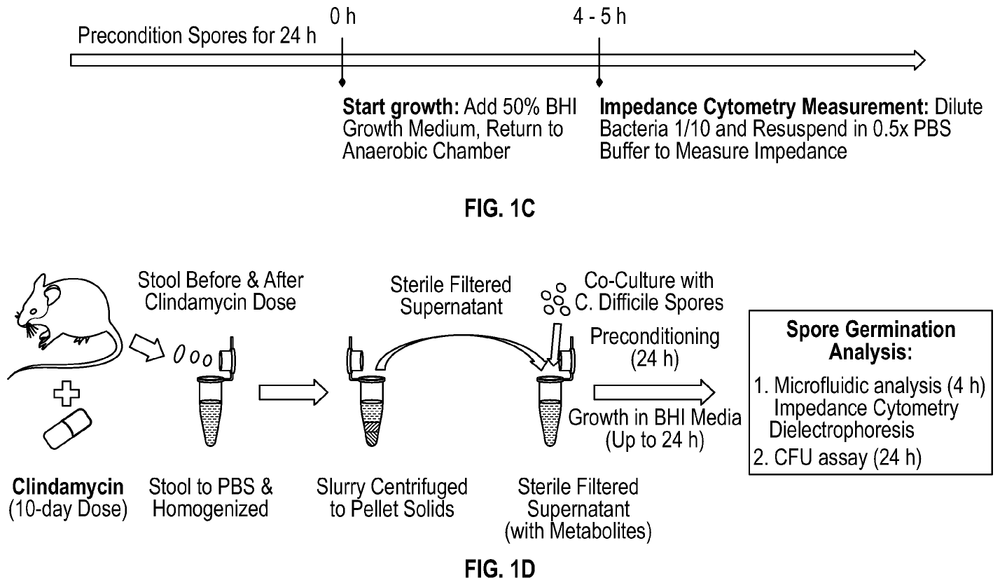

FIG. 1A shows germination of C. difficile spores to their vegetative form is inhibited by commensal bacteria in “normal” microbiota that secrete hydrolase enzymes capable of metabolizing primary bile salts into secondary bile salts, whereas antibiotic disrupted microbiota lacking commensal bacteria can exhibit higher primary to secondary bile salt levels, leading to enhanced spore germination. FIG. 1B shows an overall experimental timeline, including spore pre-conditioning, bacterial growth, microfluidic phenotypic analysis and microbiological validation, with specific sample preparation steps shown in FIG. 1C for impedance cytometry analysis. FIG. 1C shows a sequence of sample collection, preparation and analysis steps for microbiota samples from the antibiotic-treated mouse model.

A diverse community of microorganisms in the human gut work in symbiosis with their host to resist colonization by opportunistic pathogens. Loss of diversity in composition of gut microbiota, due to antibiotic administration and/or aging, is linked to the degradation of immune functions and the onset of various diseases. However, there is a need for point-of-care tools to rapidly quantify how antibiotic-induced alterations to microbiota diversity impact susceptibility of the host to colonization by pathogenic bacteria, since current metagenomic tools can only be applied within specialized laboratories. Using Clostridioides (previously Clostridium) difficile (C. difficile) infection (CDI) as a model, wherein susceptibility to this opportunistic pathogen is strongly influenced by the microbiota, this applicant discusses a rapid detection strategy for quantifying how alterations in microbiota factors affect the germination of C. difficile spores, which is the first step for CDI. CDI occurs due to C. difficile outgrowth upon interruption of the host gut microbiota, thereby causing toxin-mediated intestinal disease. While the disease can be treated by standard antibiotic regimes, a significant subset of patients (˜25%) exhibit recurrent CDI (rCDI), with 35-65% experiencing multiple episodes of rCDI. CDI recurrence is linked to the germination and outgrowth of C. difficile due to spores present from the initial infection. CDI is responsible for nearly a half million annual incidences and 29,000 fatalities in the US, as well as billions of dollars spent in combating the disease, making it the single most common cause of antibiotic-induced enteric infection and causing it to be listed as a priority within the US National Action Plan for Combating Antibiotic-Resistant Bacteria.

CDI is transmitted through ingestion of spores, which can germinate in the colon. Germination of C difficile spores is induced by primary bile salts in the small intestine of the host, such as glycine and the presence certain cholate-derived bile salts, including taurocholate, glycocholate, cholate and deoxycholate, as shown in FIG. 1A. Commensal gut microbiota can secrete hydrolase enzymes that metabolize primary bile salts into secondary bile salts, which inhibit C difficile spore germination in the colon through the action of the host microbiota. However, reduction in microbiota diversity due to antibiotic treatment prevents this conversion, thereby increasing host susceptibility to spore germination, which can lead to bacterial colonization and toxin-mediated intestinal damage. C. difficile spore germination is a complex process which is also regulated by pH, amino acids, and calcium present in the intestinal lumen. The inhibition of spore germination has been explored as a mechanism for the treatment of CDI.

The germination of C difficile spores into vegetative bacteria involves major alterations to cellular structure, machinery, and morphology. These modifications can result in characteristic electrophysiology differences between C. difficile in its spore versus vegetative form. Hence, using dielectrophoresis in homogeneous samples to characterize electrophysiological properties of C difficile in spore and vegetative forms, distinctive differences can be used to quantify spore germination in heterogeneous samples under different metabolite conditions from the host microbiota, such as by using high-throughput single-cell impedance cytometry. Dielectrophoresis (DEP) is based on translation of polarized cells within a spatially non-uniform electric field, either towards the field by positive DEP (pDEP) or away from the field by negative DEP (nDEP). It can used in this work to recognize C difficile in its vegetative form, based on its sharp transition from nDEP at low frequencies (<100 kHz) to strong pDEP at high frequencies (e.g., >1 MHz). DEP responses can been used to measure differences in bacteria based on cell wall, tumor cells based on membrane capacitance, stem cells based on differentiation lineage, quantify mitochondrial-induced cytoplasmic conductivity variations and identify exosomes derived from highly invasive pancreatic tumor cells based on their enhanced conductance due to membrane fluidity. Such electrophysiology-related differences can also form the basis for distinction of C. difficile after antibiotic and probiotic treatments.

Single-cell impedance cytometry is a label-free microfluidic technique to measure the electrical impedance of single cells flowing at high throughput (e.g., 300-400 cells per second) past electrodes patterned within a microchannel, such as by detecting the disruption in current flow under an AC electric field over a range of frequencies (e.g., 0.5-50 MHz). While the impedance at low frequencies (e.g., about 0.5 MHz) can be determined by cell volume, the impedance magnitude and phase at successively higher frequencies offer information on electrophysiology of the cell interior. Impedance cytometry cane been used to analyze various cell types, including stem cells, tumor cells, lymphocytes, and parasite-infected red blood cells. It can be thereby used to detect subtle phenotypic alterations on bacteria after interaction with clinically relevant ex vivo samples, as would be required for quantifying the germination of C. difficile spores with alterations in microbiota factors.

In vitro C. difficile spore germination can be conducted within media from an ex vivo sample containing metabolites secreted by gut microbiota, thereby characteristically altering media composition due to loss of microbiota diversity. This is simulated by gradually increasing the level of primary bile salt—taurocholate, and by using the metabolite milieu obtained from sterile filtered fecal supernatant from antibiotic-treated mice. Application of this impedance cytometry-based assay can be validated by its comparison to standard assays based on colony forming units (CFU) and the bacterial growth assay in liquid media. The interior electrophysiology of single C. difficile cells in their live vegetative form can be distinguished versus that of other C. difficile forms (spores and non-viable bacteria) by conducting the impedance cytometry at higher frequencies (e.g., about 10 MHz) in moderately conductive media (e.g., 0.5× phosphate buffered saline (PBS)). While size and impedance magnitude do not significantly differ, likely due to spore aggregation, substantive differences can be apparent in impedance phase, such as due to a higher degree of dielectric contrast arising from the insulative cell envelope (cell wall and membrane) to the conductive cell interior (cytoplasm) for vegetative C. difficile versus its spore form. This electrophysiology difference can be validated by a sharp transition from nDEP to strong pDEP behavior that is obtained only for C. difficile in its vegetative form. As a result, this single-cell electrophysiology-based method can identify and quantify spore germination within 4 hours of growth, whereas conventional methods can require 24 hours and highly concentrated sample (˜10⁹ spores/mL), as well as a specialized laboratory setting. Furthermore, this single-cell electrophysiology-based method can detect significant differences in spore germination after just four hours of incubation in the growth media containing the metabolite milieu obtained from a clindamycin-treated versus untreated mouse model, with a detection limit of about 100 germinated C. difficile cells and differences occurring due to less than 50 germinated cells between the respective samples. In this manner, by benchmarking the reported susceptibility of antibiotic-disrupted microbiota to CDI versus clinical measures of CDI in vivo within a standard mouse model, this ex vivo tool can find application in future work for screening at-risk patients to inform clinical management decisions.

### 2. Materials and Methods

### 2.1 Bacterial Strains and Sample Preparation

Clostridioides difficile strain UK1 (ribotype 027) can be used in all experiments. Vegetative cells can be generated by inoculating 10 mL of liquid BHIS (Thermo Fisher Scientific, MA, USA) media, supplemented with the appropriate taurocholate levels (typically 0.1%) or with the metabolic milieu from the stool sample of the antibiotic-treated mouse model. This can be followed by growth for 24 hours, in an anaerobic chamber (BACTRON, Sheldon Manufacturing, OR, USA). Following growth, 10 μL of culture can be streaked on BHIS agar plates supplemented with 0.1% taurocholate. Bacteria can be grown on these plates for 7 days before being washed with ice cold sterile DI water to enumerate spores. To generate the spore sample, the spores can be concentrated through centrifugation at 15,000 g for 15 minutes, and resuspended in 500 uL of 20% Histodenz (Sigma-Aldrich, USA) with an additional 500 uL of 50% Histodenz added to purify spores through gradient centrifugation at 15,000 g for 15 minutes. Spores can be centrifuged and washed 3 times with sterile DI water in order to remove any residual Histodenz. Spores can then be heated to 70° C. for 20 minutes to kill any remaining vegetative cells and stored for up to 6 months at 4° C. An example of the overall experimental timeline is shown in FIG. 1A.

### 2.2 Spore Germination Assay

In order to measure spore germination, 10 μL of spores can be suspended in PBS supplemented with 0%, 0.001, 0.01, or 0.1% taurocholate for 24 hours. Spores can then either be placed in liquid BHIS and allowed to grow for 24 hours with growth being measured every 20 minutes by a Cerillo Optoreader (Cerillo Inc., Charlottesville, Va., USA) or can be serially diluted and streaked on BHIS agar plates for CFU counts.

### 2.3 Dielectrophoresis Analysis

Dielectrophoresis analysis was performed using a 3DEP reader (e.g., DEPtech, Uckfield, UK) with a recording interval set to 30 seconds at 10 Vₚₚ, with data collected over 20 points between, for example, 50 kHz to 10 MHz. Live C. difficile in vegetative and spore form were analyzed after adjusting concentrations to an OD600 of 1.0, and resuspension in DI water, with media conductivity adjusted to 30 μS/cm (using PBS) prior to experimentation. The average relative DEP force at each frequency can be obtained by analyzing, for example, four measurements. By analyzing the spatio-temporal variations in light intensity from particle scattering, the relative DEP level at each frequency in the device can be obtained. Briefly, the 3DEP system uses specific bands of regions adjoining the gold-plated electrode stripes patterned within 20 different wells. By taking in account the electric field distribution within the wells, the system normalizes light intensity against the background at zero field time, from where the relative normalized DEP force, at each frequency, can be estimated, as previously reported. The normalized DEP spectra can be fit to multi-shell dielectric models to estimate dielectric parameters (refer to Supplementary Information (SI) Section II for further details on DEP theory and modeling).

### 2.4 Impedance Cytometry Assay

The timeline and sample preparation steps leading to impedance cytometry are shown in FIG. 1B, for acquisition using the Amphasys Z32 impedance analyzer (Amphasys AG, Switzerland). Spores conditioned in taurocholate or in fecal supernatant for 24 hours can be diluted in a 1:1 ratio in brain heart infusion supplemented (BHIS) media and allowed to germinate in growth media, for example, for 4 or 5 hours (depending on the experiment), prior to impedance analysis. The samples can then be loaded into a microfluidic chip with channels of, for example, 30 micrometers (μm) depth and 30 μm width, using a syringe pump (Pump 11 Pico Plus Elite, Harvard Apparatus, Holliston, Mass., USA) that enabled pulse-free flow at 10 μL/minute. Microfluidic channels of other depths and widths (or diameters) can be used. Impedance measurements can be performed at 500 kHz, 2 MHz, and 10 MHz using two different media conductivities at 1.6 S/m (e.g., 1×PBS) and 0.8 S/m (e.g., 0.5×PBS) (e.g., Thermo Fisher Scientific, MA, USA) for 5 minutes.

### 2.5 Statistical Analysis

Statistical analysis can be performed using Prism version 8 (GraphPad Software, San Diego, Calif.). Significance can be defined as p<0.05. Comparisons between two groups can be done using a Student's two-tailed t-test, while comparisons between multiple groups can be performed using a One-way ANOVA with a Tukey's multiple comparisons posttest or a Two-way ANOVA with Dunnett's multiple comparisons posttest.

### 2.6 Animal Work

Animal experiments were performed under a protocol approved by the Animal Institutional Care and Use Committee at the Center for Comparative Medicine at the University of Virginia, in line with national standards. Per FIG. 1C, C57BL/6J male mice, 8 weeks of age, (Jackson Laboratories, Farmington, CT, USA) can be either untreated or given clindamycin (25 mg/kg/day) for 10 days via oral gavage (n=7 animals per group). Seven days after final antibiotic administration, stool can be collected from these mice which can be pooled, homogenized, and diluted 1/10 w/v in PBS. This slurry can then be sterile filtered using a 0.2 μm syringe filter (EMD Millipore, Darmstadt, Germany). 10 μL of UK1 spores from the same stock can then be conditioned for 24 hours then diluted 1/10 for use in impedance cytometry or CFU determination assays.

### 3. Results and Discussion

### 3.1 Microbiological Analysis of Spore Germination

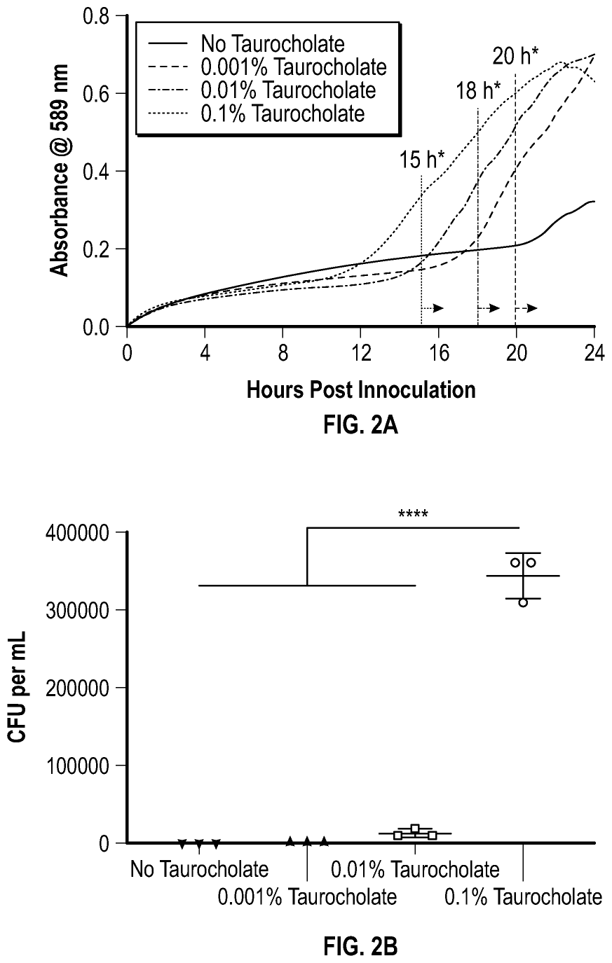

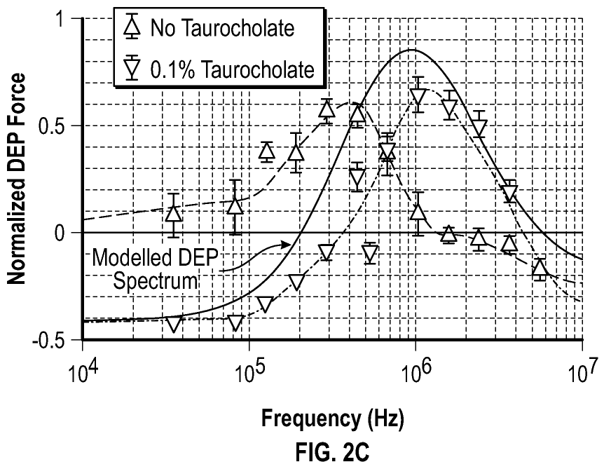

FIGS. 2A-2C show analysis following incubation of UK1 C. difficile spores in standard growth media with varying levels of taurocholate. FIG. 2A shows growth curves over 24 h show significant differences (*p<0.05) for all levels of taurocholate from varying time-points onward (marked by dashed lines). FIG. 2B shows that a CFU assay after 24 h show significant (****p<0.0001) spore germination for growth media with 0.1% taurocholate when compared with other conditions. FIG. 2C shows a chart representing DEP spectra after 4 h in growth media with versus without taurocholate show clear alterations to the average electrophysiological properties of each heterogeneous population, substantiating the presence of vegetative C. difficile in media with 0.1% taurocholate. The solid line represents the modelled behavior of a homogeneous vegetative C. difficile population, as estimated by the multi-shell dielectric model.

C. difficile spore germination is strongly dependent on the balance of primary bile salts that promote spore germination (e.g., taurocholate and glycocholate) versus secondary bile salts that inhibit germination (e.g., chenodeoxycholates). Hence, how the addition of taurocholate, at levels from 0.001-0.1%, affects C. difficile spore germination is analytically important. Per the growth measurements in liquid media (FIG. 2A), it is apparent that spore germination is progressively higher with increasing levels of taurocholate. Significant differences in optical density (compared to “no taurocholate”) due to presence of vegetative C. difficile are apparent, onward from about 20 hours for 0.001% taurocholate, from about 18 hours for 0.01% taurocholate and from about 15 hours for 0.1% taurocholate (two-way ANOVA with Dunnett's multiple comparisons posttest, *p<0.05). This trend is also apparent from the CFU analysis after 24 h in FIG. 2B, wherein significant spore germination is apparent at 0.1% taurocholate levels (****p<0.0001) and a substantial increase can be measured at 0.01% taurocholate levels. However, spore germination is not apparent at 0.001% taurocholate levels or in the absence of taurocholate. The normalized DEP force versus frequency spectra (FIG. 2C) for heterogeneous populations after 4 hours in growth media, with no taurocholate versus with 0.1% taurocholate, shows a close resemblance of the latter to the model spectra obtained from a homogeneous sample of vegetative C. difficile (further explored in the following section). Hence, presence of a vegetative sub-population within the sample conditioned in 0.1% taurocholate (FIG. 2C) based on the net electrophysiological properties of this heterogeneous sample can be inferred, consistent with the assessment of traditional assays (FIGS. 2A and 2B), but conducted in a shorter time frame (4 h versus >15 h).

### 3.2 Dielectrophoretic Analysis of Electrophysiology Differences

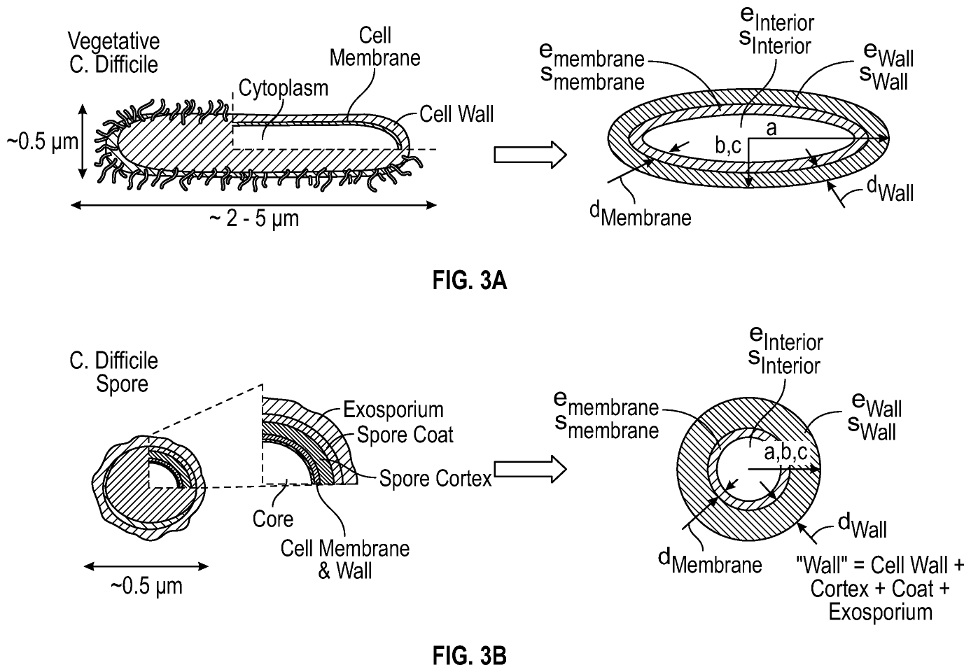

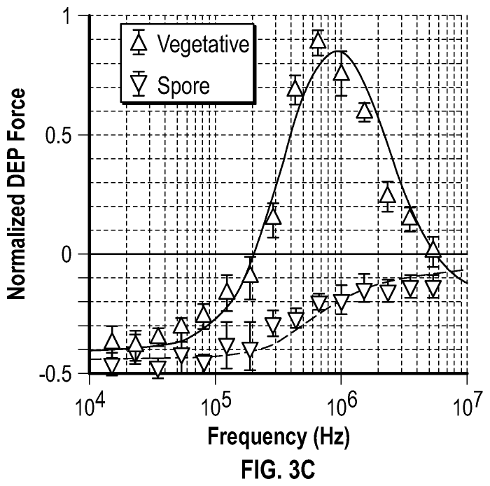

FIGS. 3A-3C show multi-shell dielectric models for C. difficile cells in vegetative form and spores are applied to the normalized DEP spectra of homogeneous populations. FIG. 3A shows multi-shell models generated based on size, shape and structural differences between vegetative cells and spores. FIG. 3B shows DEP spectra of homogenous samples of vegetative cells and spores show clear differences in polarization and electrophysiological properties of each sample type. The fits obtained from the multi-shell models are presented as solid lines.

C. difficile cells in their vegetative form present a unique morphology, with a characteristic rod-like shape, usually about 0.5 μm wide by about 2 to 5 μm long (FIG. 3A). Structurally, they are characterized by the presence of a thick cell wall (about 30 nanometers (nm)), enveloping a cell membrane (about 8 nm thick) and cytoplasm. As for C. difficile spores, their genesis process starts with an asymmetric division within the larger mother cell to generate a smaller spore that is suspended within the vegetative mother cell cytoplasm. A thick peptidoglycan layer (about 100 nm thick), known as cortex, is added upon the cell membrane and wall, helping to maintain metabolic dormancy. Further layers are built upon the spore, generating a proteinaceous coat (about 100 nm thick) which protects the spore from, for example, phagocytosis or enzymatic and oxidative reactions. The final, outermost layer, known as exosporium, is a permeable carbohydrate-based layer, likely responsible for host-pathogen interactions. At the end of this process, a round, metabolically dormant spore, usually about 0.5 μm in diameter, is formed (FIG. 3A). These morphological and structural differences between C. difficile spores and vegetative cells are translated into clear differences in their DEP spectra—for example, see FIG. 3B.

Vegetative C. difficile cells screen electric fields at frequencies below 100 kHz to exhibit nDEP behavior, due to their insulating cell envelope, but polarize under electric fields at frequencies above 1 MHz to exhibit pDEP behavior, due to the conducting cytoplasmic regions at the cell interior. In fact, this sharp transition from nDEP to pDEP behavior is a characteristic feature of their high aspect ratio ellipsoidal shape. This nDEP to pDEP transition is apparent for homogeneous samples of vegetative cells but does not occur for homogeneous spore samples. This is explained by their inability to polarize under the applied electric fields, due to the considerably thick, protective layers of spores, amounting to greater than 200 nm in total of insulating material, rendering spores effectively non-polarizable under these experimental conditions. These observations were confirmed through the implementation of multi-shell dielectric models to estimate dielectric parameters for each population (Table S1; SI Section II). C. difficile vegetative cells can be approximated to an ellipsoid including individual shells for the insulating cell wall and membrane, and the conductive cytoplasm. As for C. difficile spores, they can be approximated to a spherical particle, also including individual shells for the conductive interior, commonly labelled as spore core, and the insulating membrane and “wall”, which in this case refers to the agglomeration of the cell wall, cortex, coat and exosporium. Fitting of these models to the data (FIG. 3B) confirms the estimation of a thick insulating layer on spores (d_{“wall”}=200 nm), with a higher permittivity and much lower conductivity than that of vegetative cells: ε_{wall}—75 versus 45 and or σ_{wall}—3×10⁻⁵ versus <<1×10⁻⁷, for spores versus vegetative, respectively (Table S1 lists the estimated properties). Furthermore, spore samples analyzed after four hours of incubation in growth media with 0.1% taurocholate show this characteristic nDEP to pDEP transition (FIG. 3C) that was modelled for DEP spectra of vegetative C. difficile and experimentally measured using homogeneous vegetative C. difficile samples, as shown in FIG. 3B. This further confirms the inference of a high degree of spore germination to their vegetative form under 0.1% taurocholate conditioning. On the other hand, spore samples incubated in growth media that lacks taurocholate exhibited only a weak level of pDEP (FIG. 3C), which was not apparent over a wide frequency range. We attribute the lack of substantial pDEP in this sample to its heterogeneity, since the presence of C. difficile in various vegetative, spore and transition forms can reduce the proportion of cells exhibiting pDEP behavior and affect the ability to determine DEP spectra of each population. This same sample heterogeneity also explains the impracticality of applying multi-shell models to the data in FIG. 3C.

### 3.3 Impedance Cytometry for Spore Germination Analysis

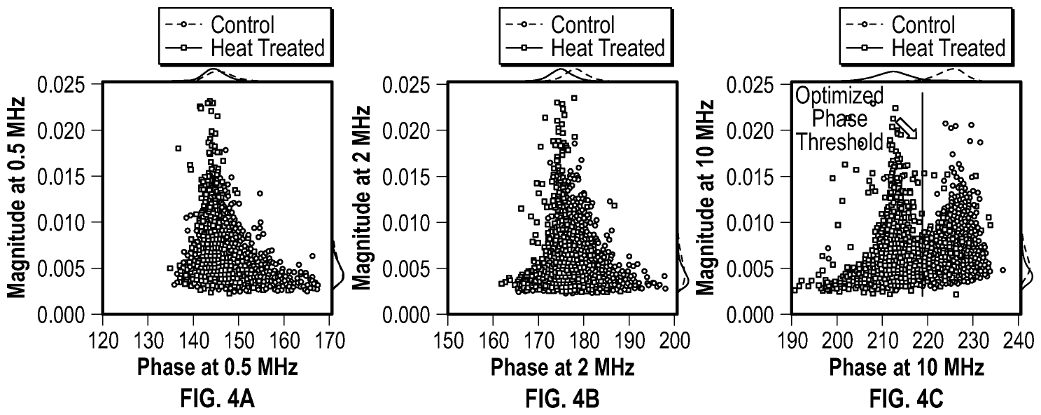

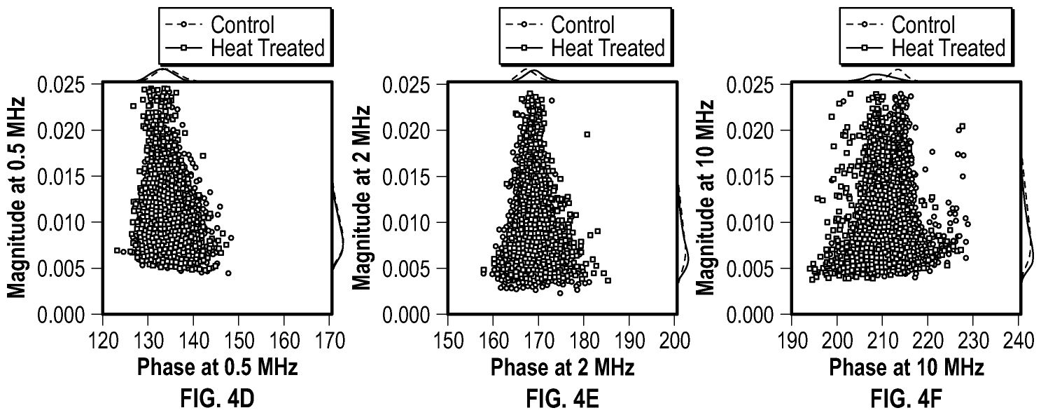

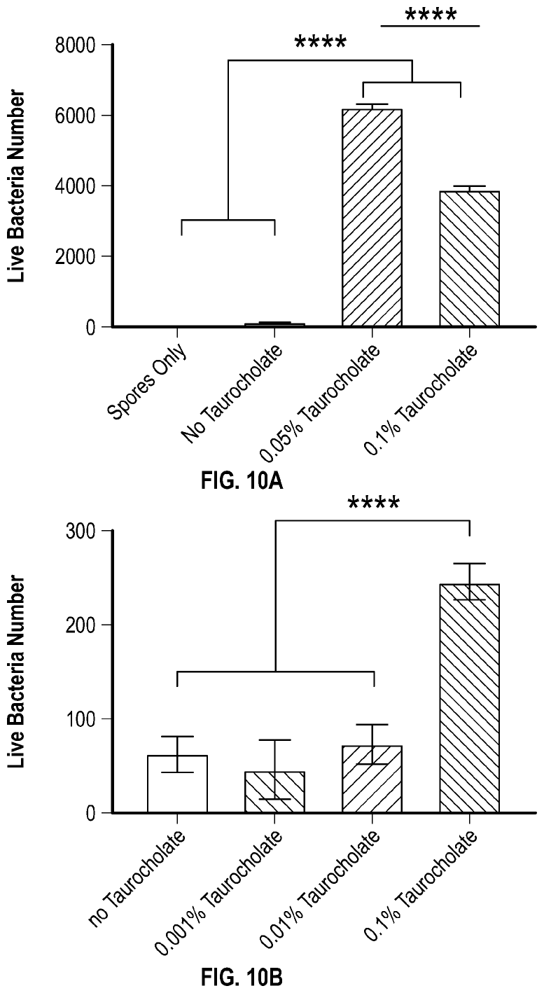

FIGS. 4A-4F show optimizing for frequency range and media conductivity for distinguishing live (or “control”) C. difficile by comparing impedance data to that of heat-treated C. difficile: FIGS. 4A-4C in 0.5×PBS and FIGS. 4D-4F in 1×PBS, acquired at 0.5 MHz (FIGS. 4A and 4D), 2 MHz (FIGS. 4B and 4E), 10 MHz (FIGS. 4C and 4F).

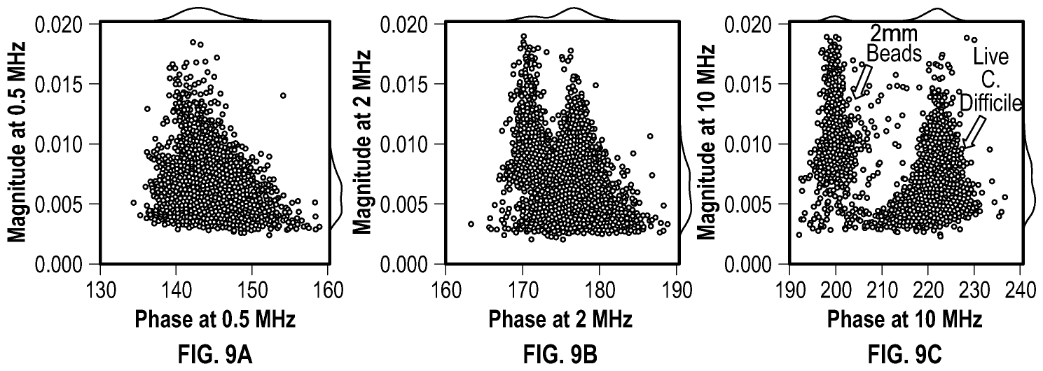

Due to sample heterogeneity during spore germination, the characteristic electrophysiology of C. difficile in its vegetative form can be used to identify germination and quantify the proportions. Hence, identifying the particular frequency range and media conductivity conditions can be used to enable gating of the impedance data corresponding to live C. difficile in its vegetative form. For this purpose, we compare impedance data on the vegetative form of live C. difficile cells to equivalently sized polystyrene beads (FIG. 9A, SI section) and to heat-treated C. difficile cells (FIGS. 4A-4F). Per the scatter plots of impedance magnitude to impedance phase in FIG. S1a, live C. difficile cells cannot be distinguished versus 2 μm polystyrene beads at 0.5 MHz, due to their size similarity. However, the greater levels of electric field penetration at higher frequencies allows for distinction based on electrophysiology of the interior, since C. difficile cells have a conductive interior versus the insulative properties of polystyrene beads. Based on FIG. 9B and FIG. 9C, acquired at 2 MHz and 10 MHz, respectively, distinction based on impedance magnitude (see histogram of Y-axis data) is still not possible, but distinction based on impedance phase (see histogram of X-axis data) seems to become apparent, especially at higher frequencies, likely due to the greater dielectric contrast. Similarly, he frequency range and media conductivity can be optimized for distinguishing live (or “control”) C. difficile by comparing impedance data to that of heat-treated C. difficile in FIGS. 4A-4F.

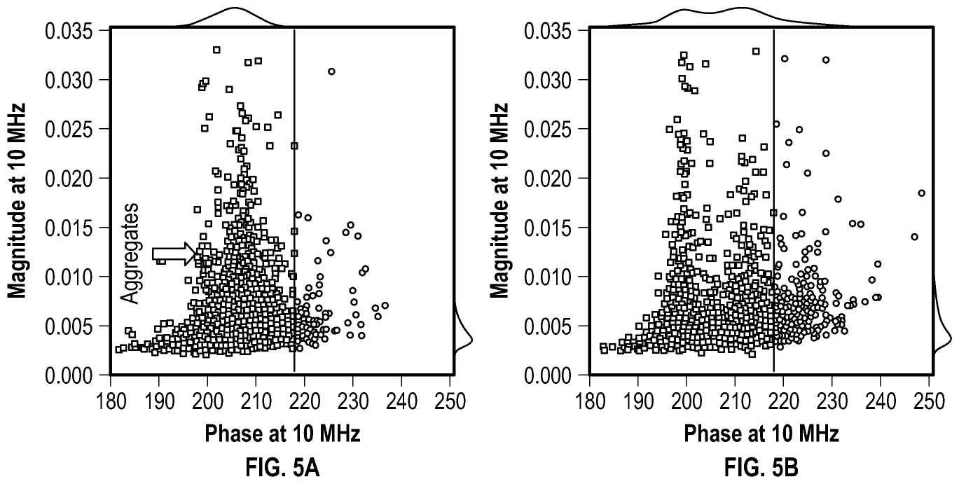

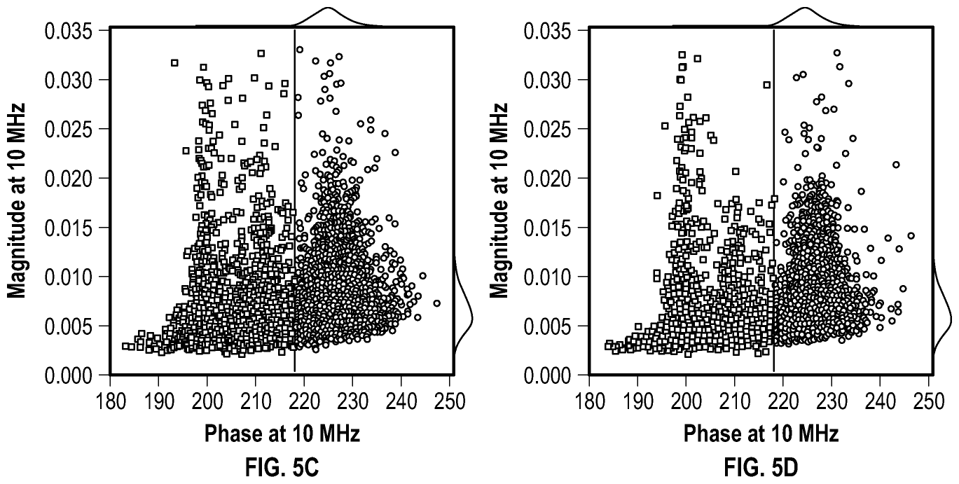

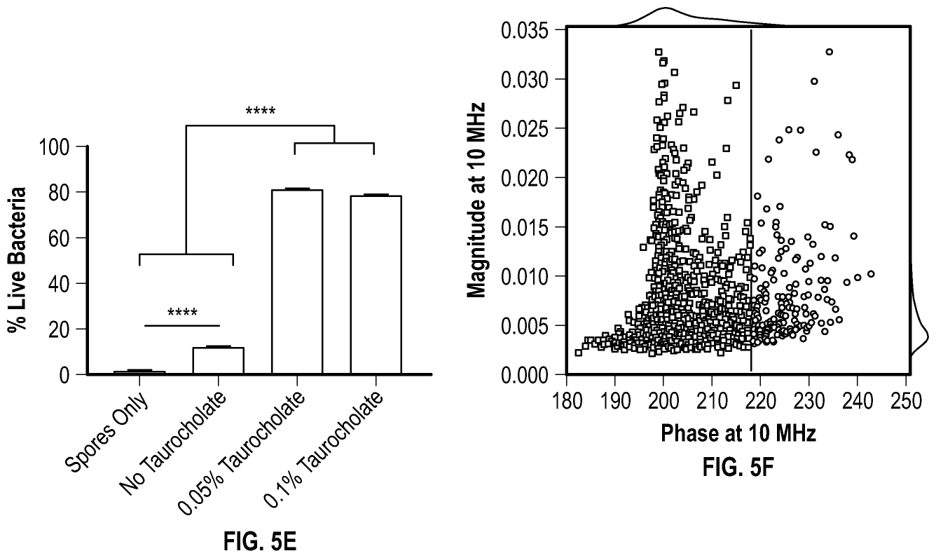

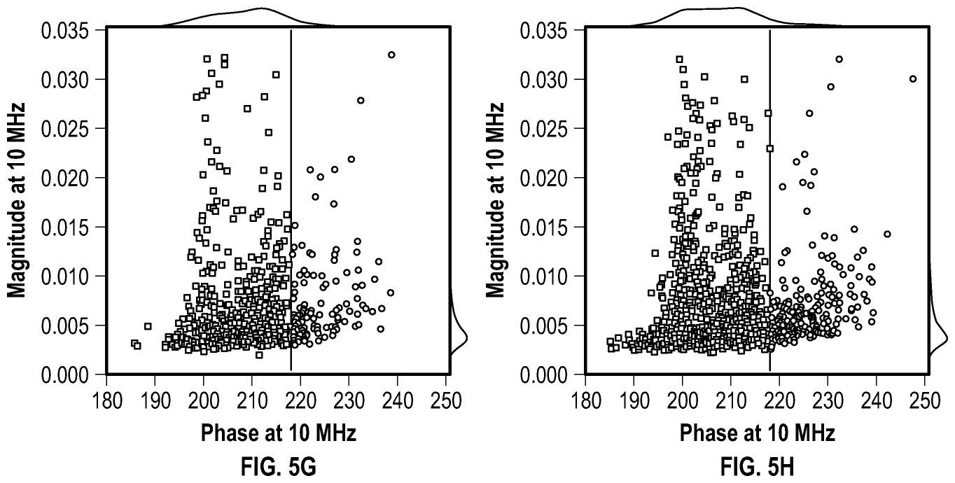

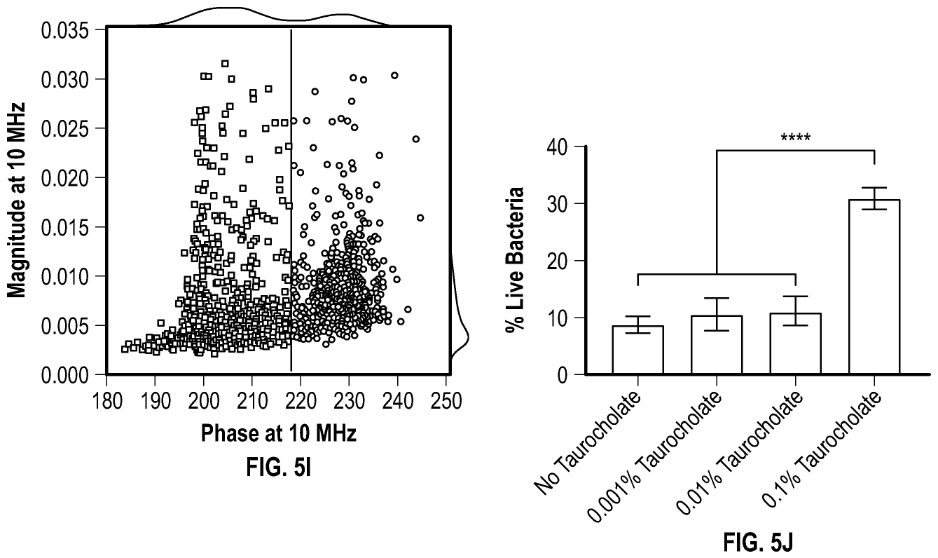

The data confirm the role of electrophysiology of cell interior in enabling an optimal level of distinction when cells are suspended in 0.5×PBS (media conductivity of 0.8 S/m) and for acquisition at 10 MHz, based on the impedance phase threshold at 218° (solid line in FIG. 4C). Using these optimized parameters (samples in 0.5×PBS, with impedance acquired at 10 MHz), the determined phase threshold is capable of identifying germinated spores can be investigated based on their purported electrophysiological resemblance to live C. difficile cells in their vegetative form. Specifically, as shown FIG. 5, it is determined whether the phase threshold is capable of identifying the increased levels of spore germination with increasing presence of taurocholate in the growth media and with increasing growth time, thereby characterizing the dynamic range of this assay.

The histogram in the “live” C. difficile phase region can be used to quantify the percentage of live bacteria in the sample in FIGS. 5E and 5J, with the respective raw number of bacteria in the sample indicated in FIGS. 9A and 9B (Supplementary Information), respectively. Based on this, it is apparent that spore germination, as determined through the phase threshold, increases with taurocholate levels and incubation time in the growth media, with germination rates of greater than 80% (4000-6000 vegetative C. difficile cells per the measured 50 micro Liter (μL) sample) after 5 h of incubation in the growth media with 0.05-0.1% taurocholate. Upon lowering to 4 h incubation in growth media with 0.1% taurocholate, germination rates drop to ˜30% (˜250 vegetative C. difficile cells per the measured 50 μL sample). Hence, the assay shows a good degree of sensitivity to variations in incubation time and proportion of primary bile salts (taurocholate) in the growth media. It is noteworthy that spores exhibit a good degree of aggregation, as apparent from the linear clustering on impedance magnitude data, thereby highlighting the need for impedance phase data for distinction of vegetative versus spore C. difficile.

FIGS. 5A-5J show applying an impedance phase threshold (acquisition at 10 MHz with sample in 0.5×PBS) for live C. difficile cells to quantify C. difficile spore germination after 5 h (FIG. 5A-FIG. 5E) and 4 h (FIG. 5F-FIG. 5J) of incubation, using spore samples under indicated conditions: (FIG. 5A) in absence of growth media versus in presence of growth media containing taurocholate at indicated levels: (FIG. 5B) 0%; (FIG. 5C) 0.05%; (FIG. 5D) 0.1%. (FIG. 5E) Summary plot with variation in percentage of detected live C. difficile for each experimental condition. The analogous experiment conducted after 4 h of spore incubation in growth media containing taurocholate at indicated levels: (FIG. 5F) 0%; (FIG. 5G) 0.001%; (FIG. 5H) 0.01%; (FIG. 5I) 0.1%; and (FIG. 5J) summary plot.

3.4 Validation with Ex Vivo Microbiota Samples

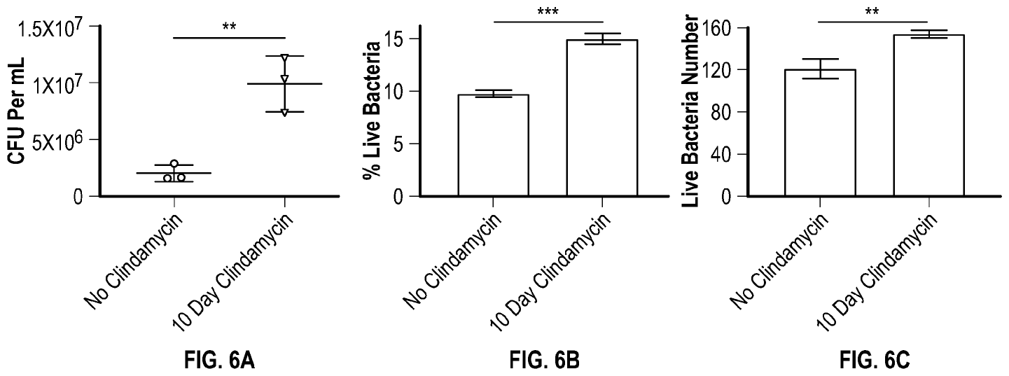

FIG. 6 shows C. difficile spore germination after culture with ex vivo microbiota samples from untreated versus antibiotic-treated mouse model to compare: (FIG. 6A) the CFU assay after 24 h of bacterial growth; to impedance assay for (FIG. 6B) percentage of bacteria and (FIG. 6C) total numbers of bacteria after 4 h of growth. (**p<0.01, ***p<0.001)

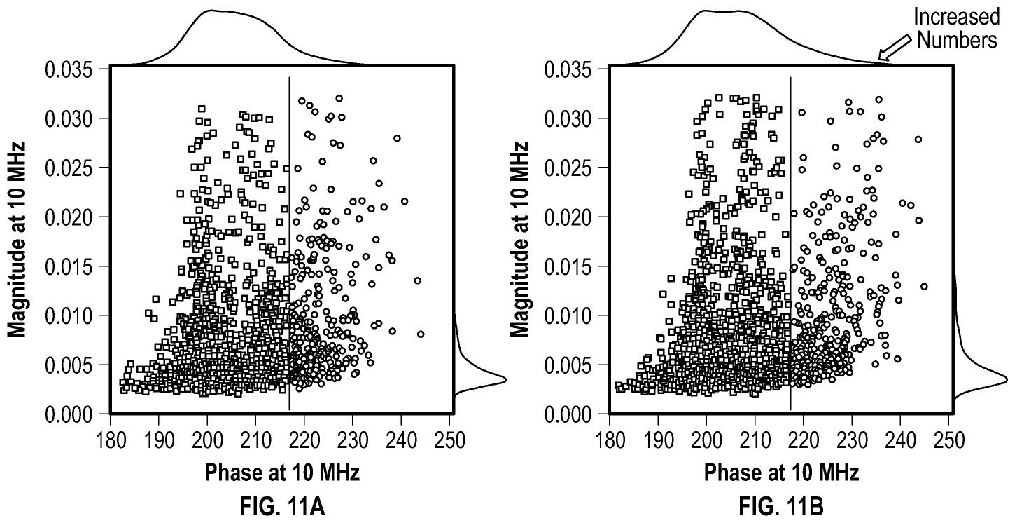

In order to validate the reported assay with realistic samples, we use ex vivo microbiota samples obtained from a mouse model, before and after 10 days of antibiotic treatment with clindamycin, as per the procedure in FIG. 1D. Upon culture of C. difficile spores with ex vivo microbiota samples obtained from untreated versus antibiotic-treated mouse model, the data from CFU analysis (FIG. 6A) shows significantly higher levels (**p<0.01) of spore germination after 24 h of growth for clindamycin-treated microbiota, based on the numbers of vegetative C. difficile, whereas spore germination is significantly lower for the untreated microbiota. Hence, a reduction in microbiota diversity after antibiotic treatment likely enhances C. difficile germination rate. In comparison, impedance analysis applied to the respective samples after just 4 h of growth (FIGS. 6B and 6C) starts to show significant differences in the percentage (***p<0.001) and total numbers (**p<0.01) of vegetative C. difficile (scatter plots in FIGS. 11A and 11B, Supplementary Information). The detection system could identify and quantify down to about 125 vegetative C. difficile cells (per 50 μL). Furthermore, unlike the case for in vitro samples wherein differences between the studied groups was higher due to the ability to effectively lower spore germination of “control” samples by eliminating taurocholate (FIG. 5 and FIG. S2), the differences in these ex vivo samples was far smaller, since both treatment groups (untreated and antibiotic-treated) exhibit spore germination, but at differing rates. Nevertheless, the detection system could distinguish differences of as few as 50 vegetative C. difficile cells (per 50 μL) between the respective treatment groups. This higher level of sensitivity allows the assay to detect differences in susceptibility of microbiota to C. difficile spore germination at far earlier times versus CFU methods (4 h versus 24 h).

### 4. Conclusions and Outlook

A single-cell electrophysiology-based detection system for identifying the phenotype of C. difficile in its vegetative form can be applied to quantify the effect of microbiota factors on germination of C. difficile spores, as obtained by varying the proportion of primary bile salts and the metabolite milieu associated with antibiotic treatment. To identify the distinctive phenotype of C. difficile in its spore versus vegetative form, a dielectric shell model can be used for fits of dielectrophoretic spectral data obtained from homogeneous samples to demonstrate the distinctively higher interior conductivity of the vegetative C. difficile form. To quantify vegetative C. difficile at high throughput and within heterogeneous samples based on this electrophysiology difference, a single-cell impedance cytometry can be optimized in frequency of electric field and media conductivity, to detect differences in the cell interior. This can be achieved by comparing impedance data on live C. difficile cells to equivalently sized polystyrene beads and to heat-treated C. difficile cells. As a result, it can be determined optimal distinction based on acquisition in 0.5×PBS (0.8 S/m) at 10 MHz, wherein impedance phase levels greater than 218° correspond to live C. difficile cells in vegetative form. Using this threshold on the impedance data, the spore germination assay shows a high degree of sensitivity to variations in incubation time in growth media and to the proportion of primary bile salts (taurocholate) in the growth media. This is apparent from a dynamic range of 6000 vegetative C. difficile cells per the measured 50 μL sample after 5 h of incubation in the growth media with 0.05-0.1% taurocholate, to −250 vegetative C. difficile cells per the measured 50 μL sample after 4 h incubation in growth media with 0.1% taurocholate. Upon validation by C. difficile spore co-culture with untreated versus antibiotic disrupted microbiota, the impedance-based assay is determined to be capable of detecting differences in susceptibility of microbiota to C. difficile spore germination. Due to the ability to measure ˜100 vegetative C. difficile cells (per 50 μL) and distinguish differences between the respective treatment groups of as few as 50 vegetative C. difficile cells (per 50 μL), the assay performs well at far earlier detection times (4 h) versus traditional CFU assays (24 h).

The assays discussed here can be used with lower spore numbers in the starting samples, since CDI can occur due to ingestion of as few as 100 spores. The assays can also be benchmarked versus clinical measures of CDI to detect susceptibility differences arising from temporally varying diversity of gut microbiota under antibiotic treatment. Based on the high detection sensitivity, speed, and the limited sample preparation needs of this assay, the systems and methods of this disclosure can be used in point-of-care settings for screening at-risk patients to inform clinical management decisions on susceptibility to initial and recurrent C. difficile infection.

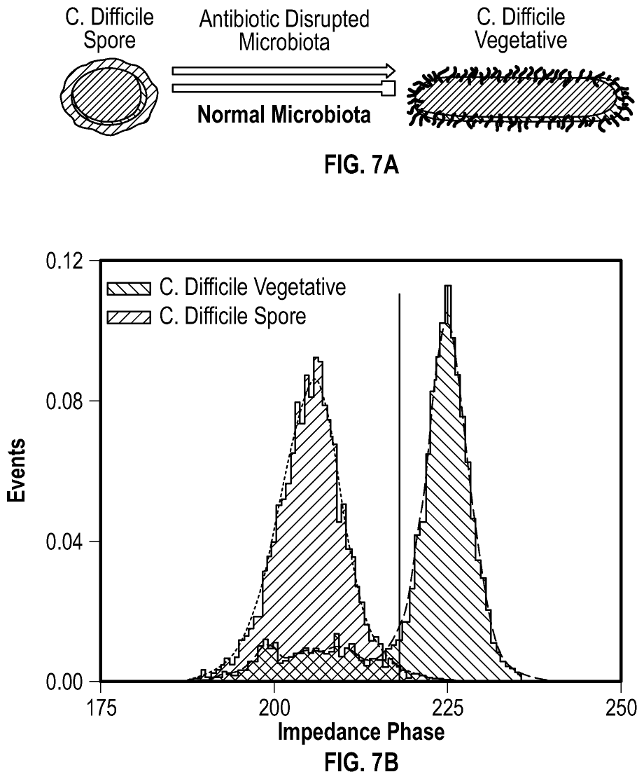

FIG. 7A shows a timeline of vegetation from C. difficile spore to C. difficile in a vegetative state following disruption of microbiota, where the spore and the vegetative bacteria include one or more physical differences that can be detected via an impedance phase change using impedance cytometry through a microfluidic device. FIG. 7B shows an example chart of plotted samples where most of the C. difficile spores and C. difficile in a vegetative state are at different impedance phases, allowing C. difficile spores and C. difficile in a vegetative state to be counted. While the phase overlap of C. difficile spores and C. difficile in a vegetative state is about 5%, the analysis is unlikely to yield false positives due to the disparity of phase shift of C. difficile spores and C. difficile in a vegetative state.

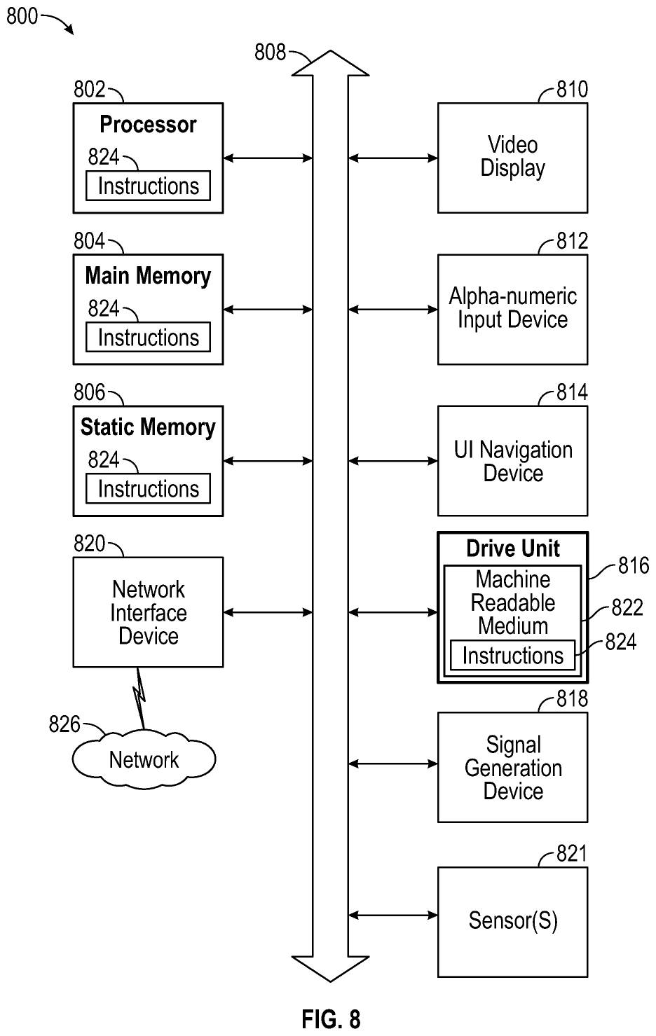

FIG. 8 is a block diagram illustrating an example of a machine upon which one or more aspects of embodiments of the present invention can be implemented. An aspect of an embodiment of the present invention can include, but is not limited thereto, a system, method, and computer readable medium that provides: quantifying bacterial spore germination by single-cell impedance cytometry for assessment of host microbiota susceptibility to infection, which illustrates a block diagram of an example machine 800 upon which one or more embodiments (e.g., discussed methodologies) can be implemented (e.g., run).

Examples of machine 800 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 800) and software architectures that can be deployed in example embodiments.

In an example, the machine 800 can operate as a standalone device or the machine 800 can be connected (e.g., networked) to other machines.

In a networked deployment, the machine 800 can operate in the capacity of either a server or a client machine in server-client network environments. In an example, machine 800 can act as a peer machine in peer-to-peer (or other distributed) network environments. The machine 800 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 800. Further, while only a single machine 800 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 802 (e.g., a central processing unit (CPU), a graphics processing unit (GPU) or both), a main memory 802 and a static memory 806, some or all of which can communicate with each other via a bus 808. The machine 800 can further include a display unit 810, an alphanumeric input device 812 (e.g., a keyboard), and a user interface (UI) navigation device 811 (e.g., a mouse). In an example, the display unit 810, input device 817 and UI navigation device 814 can be a touch screen display. The machine 800 can additionally include a storage device (e.g., drive unit) 416, a signal generation device 818 (e.g., a speaker), a network interface device 820, and one or more sensors 821, such as a global positioning system (GPS) sensor, compass, accelerometer, or other sensor.

The storage device 816 can include a machine readable medium 822 on which is stored one or more sets of data structures or instructions 824 (e.g., software) embodying or utilized by any one or more of the methodologies or functions described herein. The instructions 824 can also reside, completely or at least partially, within the main memory 802, within static memory 806, or within the processor 802 during execution thereof by the machine 800. In an example, one or any combination of the processor 802, the main memory 802, the static memory 806, or the storage device 816 can constitute machine readable media.

While the machine readable medium 822 is illustrated as a single medium, the term “machine readable medium” can include a single medium or multiple media (e.g., a centralized or distributed database, and/or associated caches and servers) that configured to store the one or more instructions 824. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 824 can further be transmitted or received over a communications network 826 using a transmission medium via the network interface device 820 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### Supplementary Information (SI) Section

Quantifying bacterial spore germination by single-cell impedance cytometry for assessment of host microbiota susceptibility to Clostridioides difficile infection.

FIGS. 9A-9C show distinction of live C. difficile in its vegetative form versus co-flowing 2 μm polystyrene beads, in 0.5×PBS buffer, using impedance cytometry acquired at: (FIG. 9A) 0.5 MHz, (FIG. 9B) 2 MHz; and (FIG. 9C) 10 MHz. FIGS. 10A-10B show a comparison of numbers of vegetative C. difficile after: (FIG. 10) 5 h in growth media, and (FIG. 10B) 4 h in growth media. (****p<0.0001). FIGS. 11A-11B show impedance data after co-culture with untreated versus antibiotic-treated microbiota. FIGS. 9A-11B are discussed together below.

S2: Fitting of DEP spectra to multi-shell dielectric models

S2.1 Theoretical Background

AC electrokinetics can be employed to induce movement of particles subjected to an AC electric field. If the AC electric field is spatially non-uniform, dielectrophoresis (DEP) exerts a frequency-selective force, causing movement of the cell. The time averaged DEP force ( [symbol] F_{DEP} [symbol] ) on a particle is given by:

[symbol]

F_{DEP} [symbol] =2πε_{medium} r³ Re[\tilde{f}_{CM}]∇|E|² Eq. (S1)

where r is the radius of the particle, Re [\tilde{f}_{CM}] the real part of the Clausius-Mossotti factor and E the electric field. When the electric field is uniform, the gradient of the magnitude of the field is zero (∇|E|²=0), resulting in zero DEP force.

The Clausius-Mossotti factor (\tilde{f}_{CM}) is a frequency-dependent measure of cell polarizability dictating force direction, given by:

$${\tilde{f}}_{C M} = \frac{{\tilde{ε}}_{particle} - {\tilde{ε}}_{medium}}{{\tilde{ε}}_{particle} + 2 {\tilde{ε}}_{medium}} \qquad Eq . ( S2 )$$

For particles more polarizable than the medium, i.e., Re [\tilde{f}_{CM}]>0, positive DEP (pDEP) ensues, with the particles moving to areas of higher electric field strength. When Re[\tilde{f}_{CM}]<0, particles are in turn less polarizable than the medium, thus negative DEP (nDEP) occurs, with particles being repelled from the regions of high field strength.

\tilde{ε}_{medium} and \tilde{ε}_{particle} are the complex permittivities of the medium and particle, respectively, and represent the frequency dependence of the material permittivity, and are given by:

$${\tilde{ε}}_{m e d i u m} = {ε}_{0} {ε}_{m e d i u m} - i \frac{{σ}_{medium}}{ω} \qquad Eq . ( S3 )$$

$${\tilde{ε}}_{particle} = {ε}_{0} {ε}_{particle} - i \frac{{σ}_{particle}}{ω} \qquad Eq . ( S4 )$$

where ε₀ is the constant vacuum permittivity (8.85×10⁻¹² F m⁻¹), ε_{medium} and ε_{particle} are the dimensionless numbers referring to the relative permittivity of the medium and particle, respectively, and σ_{medium} and σ_{particle} are the conductivities (S m⁻¹) of the medium and particle, respectively. Moreover, the r³ term means the time averaged force is proportional to particle volume.

### S2.2 Quantification of the DEP Force

DEP spectral measurements were conducted on a 3DEP dielectrophoretic analyzer (DepTech, UK). A non-uniform AC field is applied to gold-plated electrodes patterned on the walls of individual chambers. The analyzer tracks the velocity of cells within individual analysis chambers to quantify the DEP force enacting on cells [3], [4]. By analyzing spatio-temporal variations in light intensity from particle scattering (after normalizing against the background at zero field−∇|E|²=0), the relative DEP force at each frequency is obtained. The DEP force is measured at different frequencies (from approximately 50 kHz to 10 MHz) applied independently within separate chambers. The trajectory of cells within each chamber is recorded at high frame rate. For a particle of mass m, the dielectrophoeretic force F_{DEP} on an accelerated particle of radius r, within a medium of viscosity η, can be determined by tracking its displacement x as a function of time t to obtain (dx/dt) and (d²x/dt²) for each population analyzed. Thus, the relative DEP force can be approximated using:

$${F}_{D E P} - 6 πη r \frac{d x}{d t} = m \frac{{d}^{2} x}{d {t}^{2}} \qquad Eq . ( S5 )$$

### S2.3 Multi-Shell Dielectric Modelling

For a mixture of a particle (e.g., mammalian cell or bacterium) suspended in a conductive medium, dielectric theory can be implemented to estimate the dielectric properties of the particle using Maxwell's mixture theory (MMT). For the specific case of a non-spherical particle, such as vegetative C. difficile, the ellipsoidal model is used, as represented in FIG. 4A. In this model, the rod-like morphology of a as vegetative C. difficile bacterium can be approximated to a prolate spheroid with semi-axes n=a, b, c defined as a>b=c. For the case of a C. difficile spore, a spherical model is used instead (FIG. 4A), where all semi-axes are considered equal (a=b=c). For both C. difficile spore and vegetative bacteria, their intricate structure is approximated to that of a two-shell model, which assumes a thick cell wall and cell membrane surrounding their internal content. In such model, there are three dispersions corresponding to interfaces between each layer, i.e., medium/wall (subscripts 3 & 2), wall/membrane (subscripts 2 & 1) and membrane/interior (subscripts 1 & 0).

Due to the configuration of the 3DEP chambers and electrodes, the electric field causes ellipsoidal particles to orient with their major axis a parallel to the electric field (x direction) [11]. Thus, the DEP force acting on an ellipsoidal particle, such as a vegetative C. difficile bacterium, is given by:

[symbol]

F_{DEP} [symbol] =2πε_{medium} abc Re[\tilde{K}_{a,3/2}]∇|E|² Eq. (S6)

where \tilde{K}_{a,3/2} is the dipole coefficient (analogous to the \tilde{f}_{CM}) along the major axis a at the final interface (medium/wall). This coefficient includes all the existing interfaces up to that point, and depends on the depolarizing factor A_{a,3/2} and the complex permittivity up to that interface. It is calculated by:

$${\tilde{K}}_{a , 3 / 2} = \frac{{\tilde{ε}}_{2 / 1} - {\tilde{ε}}_{3}}{3 ( {A}_{a , i} ( {\tilde{ε}}_{2 / 1} - {\tilde{ε}}_{3} ) + {\tilde{ε}}_{3} )} \qquad Eq . ( S7 )$$

$${A}_{a , 3 / 2} = \frac{1}{1 - {q}^{2}} + \frac{q}{{( 1 - {q}^{2} )}^{3 / 2}} arcos ( q ) \qquad Eq . ( S8 )$$

where q is the ratio of semi-axis (a₂/b₂), while the complex permittivity up to each interface (for i=1 and 2) is calculated through [12]:

$${\tilde{ε}}_{i / i - 1} = {\tilde{ε}}_{i} \frac{2 {\tilde{ε}}_{i} + {\tilde{ε}}_{i - 1} - 2 {υ}_{i} ( {\tilde{ε}}_{i} - {\tilde{ε}}_{i - 0} )}{2 {\tilde{ε}}_{i} + {\tilde{ε}}_{i - 1} + {υ}_{i} ( {\tilde{ε}}_{i} - {\tilde{ε}}_{i - 0} )} \qquad Eq . ( S9 )$$

where vᵢ is the volume fraction at the i-th layer (for i=1 and 2) calculated as vᵢ=(1−dᵢ/a₂)³, with dᵢ being the thickness of the i-th layer (dᵢ<<a₂).

For the case of a spherical particle, such as a C. difficile spore, it is assumed to randomly orient within the 3DEP chamber. Thus, for spherical particles (a=b=c), the DEP force is dependent on dipole coefficients for all axes, i.e., Eq. S6 reverts to Eq. S1, where \tilde{f}_{CM}, is considered instead and calculated as:

$${\tilde{f}}_{C {M}_{3 / 2}} = \frac{{\tilde{ε}}_{2 / 1} - {\tilde{ε}}_{3}}{( {\tilde{ε}}_{2 / 1} - {\tilde{ε}}_{3} ) + {\tilde{ε}}_{3}} \qquad Eq . ( S10 )$$

By calculating Re[\tilde{K}_{a,3/2}] or Re[\tilde{f}_{CM}], it is possible to model the corresponding DEP behavior of the particles by generating multiple polarization dispersions and fitting them to the experimental DEP data. The dispersion with the optimal fit thus gives the estimate of dielectric properties for a specific sample. For the modelling process, the values of σ_{medium} and ε_{medium} are fixed at 10 mS m⁻¹ and 78, respectively. Membrane thickness was fixed based on literature values at 8 nm, while wall thickness was also fixed but varied with sample type: 30 nm for vegetative C. difficile and 200 nm for spores. The fitted parameters from FIG. 3, are summarized in Table S1, which shows two-shell model-derived dielectric parameters for C. difficile in vegetative and spore form, where Square brackets denote units; 1 indicates dimensionless parameter.

| | | | | | | | Dimension [m] | | | |
|---|---|---|---|---|---|---|---|---|---|---|
| | Permittivity [1] | | | Conductivity [S/m] | | | Major | Minor | Thickness [m] | |
| | Wall | Membrane | Interior | Wall | Membrane | Cytoplasm | axis | axis | Wall | Membrane |
| Sample | (ε_{wall}) | (ε_{membrane}) | (εᵢₙₜₑᵣᵢₒᵣ) | (σ_{wall}) | (σ_{membrane}) | (σᵢₙₜₑᵣᵢₒᵣ) | (a) | (b,c) | (d_{wall}) | (d_{membrane}) |
| Vegetative | 4. | 15 | 60 | 3.0 × 10⁻⁵ | 1.0 × 10⁻⁷ | 0.45 | 2.8 × 10⁶ | 2.5 × 10⁻⁷ | 3.0 × 10⁻⁸ | 8.0 × 10⁻⁹ |
| Spore | 75 | 15 | 60 | 1.0 × 10⁻⁷ | 1.0 × 10⁻⁷ | 0.45 | 2.8 × 10⁻⁷ | 2.6 × 10⁻⁷ | 2.0 × 10⁻⁷ | 8.0 × 10⁻⁹ |

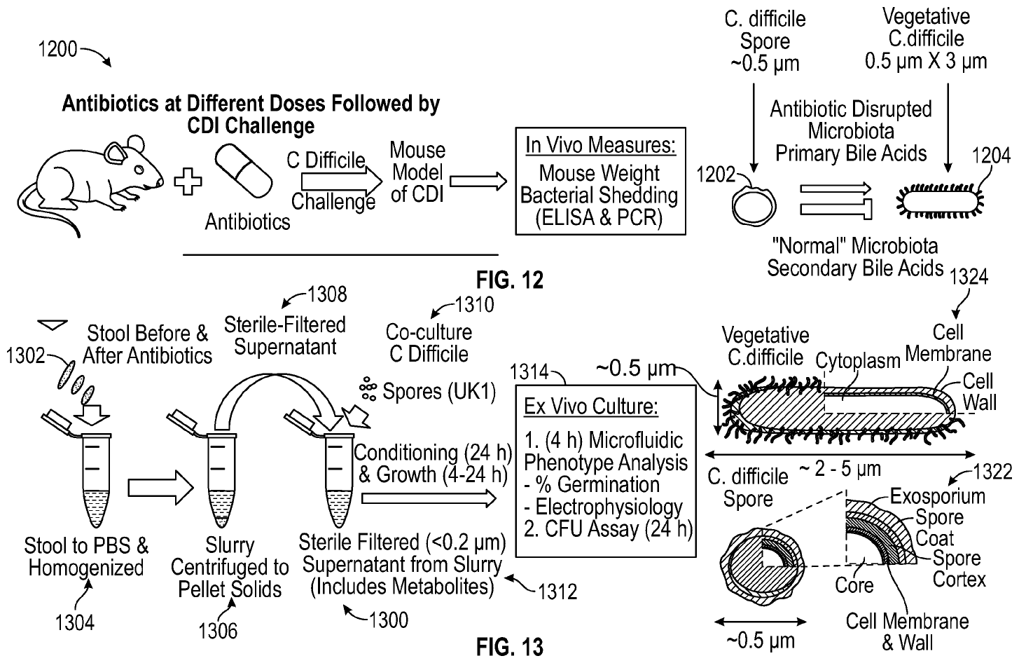

FIG. 12 illustrates a schematic view of an in vivo mouse model process for determining an effect of antibiotics on a microbiota of a mouse. For example, the mouse model process 1200 can include steps of treating mice with antibiotics, such as clindamycin, which can be given at, for example, 25 mg/kg/day for 10 days via oral gavage (n=7 animals per group). Seven days after final antibiotic administration, stool can be collected from these mice which can be pooled, homogenized, and diluted 1/10 w/v in PBS. This slurry can then be sterile filtered using a 0.2 μm syringe filter. 10 μL of UK1 spores from the same stock can then be conditioned for 24 hours then diluted 1/10 for use in impedance cytometry or CFU determination assays, such as for comparison with the ex vivo process discussed above and below.

FIG. 12 also shows C. difficile in a spore state 1202 and C. difficile in a vegetative state 1204 where the C. difficile in the spore state can have a more rounded shape and the C. difficile in the vegetative state 1204. FIG. 13 shows additional details of C. difficile in a spore state 1322 and C. difficile in the vegetative state 1324.

FIG. 13 also shows a method 1300 of to assess spore germination by using high throughput single-cell impedance cytometry (for example, at 300-400 events per second) to quantify live bacterial cells. Sampling at 300-400 events per second to help maximize a number of cells per second that can be accurately measured while still limiting coincident measurement. Quantification can be performed by gating for characteristic electrophysiology of vegetative bacteria versus spores, so that germination can be assessed after four hours of culture based on a detection limit of about 100 live cells. To detect the phenotype of live C. difficile bacteria, characteristically higher cytoplasmic conductivity can be used versus that of spore aggregates and non-viable C. difficile forms, which causes a distinctive high-frequency (e.g., 10 MHz) impedance phase dispersion within moderately conductive media (0.8 S/m). In this manner, significant differences in spore germination rates can be detected within just 4 hours, with increasing primary bile salt levels in vitro and using ex vivo microbiota samples from an antibiotic-treated mouse model to assess susceptibility to C. difficile infection. This method can be used as a rapid diagnostic tool for assessing host microbiota susceptibility to bacterial colonization after key antibiotic treatments.

For example, as shown in FIG. 13, the process 1300 can be a method for predicting in vivo colonization and infection dynamics by quantifying bacterial spore germination in an ex vivo assay. The method can include creating an ex vivo assay including bacteria spores and a homogenized stool sample from an in vivo model, which can include a step of collecting stool before and after antibiotics 1302 and the stool can be introduced into a phosphate buffered saline (PBS) and the mixture can be homogenized 1304. At step 1306, the mixture or slurry can be centrifuged to separate stool solids. At step 1308, the supernatant can be sterile-filtered from the slurry. At step 1310, the bacteria spores can be co-cultured with the sterile-filtered supernatant. At step 1312, the bacteria spores in taurocholate or in fecal supernatant can be conditioned. Optionally, the process can include germinating the bacteria spores in growth media.

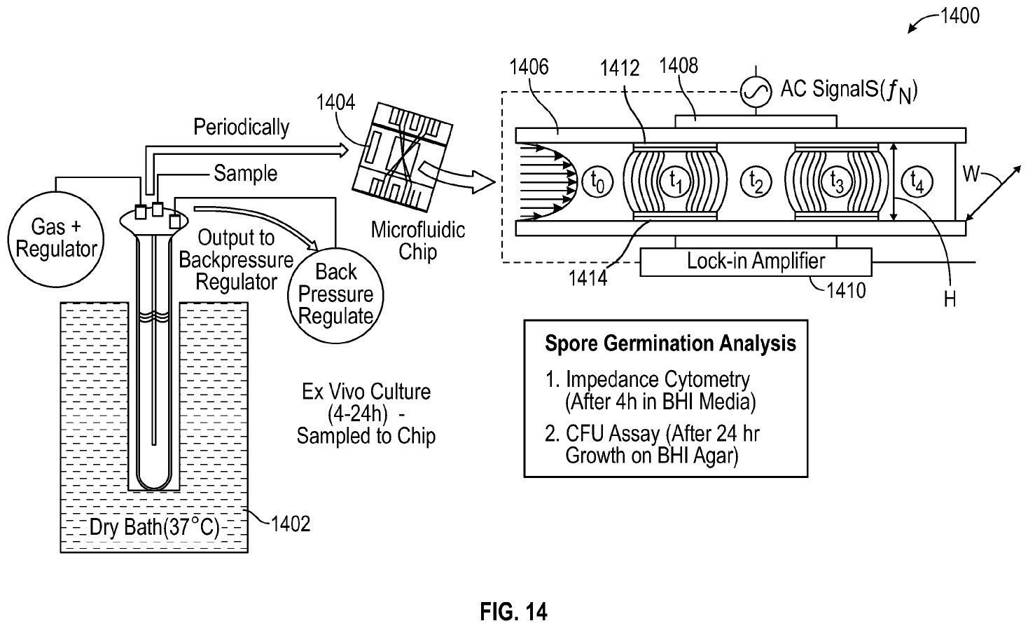

FIG. 14 illustrates a schematic view of a process 1400, which can be a continuation of the processes 1200 or 1300 or can be a standalone process. The ex vivo assay or culture can be placed into a dry bath 1402, which can be optionally controlled by a gas regulator, a back-pressure regulator, and one or more additional valves. The culture can be sampled, optionally periodically. The sample(s) or ex vivo assay can be loaded into a microfluidic chip 1404, such as by using microfluidic pneumatic systems to draw a small sample from the ongoing ex vivo spore germination culture to assay vegetative numbers by impedance cytometry. The samples can be drawn at regular intervals or optionally other irregular intervals. The microfluidic chip 1404 can define one or more microfluidic channels 1406. The channel(s) 1406 can be detection channels having a depth or height H of 30 μm and a width W of 30 μm. The microfluidic chip 1404 can include one or more signal generation devices 1408 and a lock-in amplifier 1410, or other amplifier configured to receive the signal from the generation device 1408.

The microfluidic chip 1404 can be used to detect vegetative bacteria and the bacteria spores by sampling the mixture in the microfluidic chip 1404 chip using impedance cytometry. The impedance cytometry can optionally be single-cell impedance cytometry. Single-cell impedance cytometry is a label-free microfluidic technique to measure the electrical impedance of single cells flowing at high throughput (300-400 cells/s) past electrodes 1412 and 1414 patterned within the microchannel 1406, such as by detecting the disruption in current flow under an AC electric field over a range of frequencies (e.g., 0.5-50 MHz). Optionally, the impedance cytometry is conducted at 500 kHz, 2 MHz, and 10 MHz, and wherein a flowrate of cells through a sampling channel of the microfluidic chip is between 300 and 400 cells per second.

Detection can include detecting an impedance phase for each cell sampled in the microfluidic chip and recording a number of events within an impedance phase range based on the detected impedance phase for each cell. The recorded events can be grouped based on an impedance phase of each event. Following detection, the vegetative bacteria and the bacteria spores can be used to predict colonization of the bacteria of the in vivo model based on the detected vegetative bacteria and bacteria spores. That is, the vegetative bacteria detected in the ex vivo assay can be counted and used to determine whether the in vivo model is sufficiently disrupted to support vegetation of C. difficile.

Though this application discusses detection of C. difficile in a spore or vegetative state, the methods discussed herein can be used to detect other bacteria using the same or similar methods, including other strains of C. difficile among various other bacteria known to cause infections.

Also, some strains or types of C. difficile can be non-toxigenic and can be beneficial to supporting a healthy and diverse gut microbiota. The presence or viability of such strains of C. difficile can therefore be indicators of a balanced microbiota or can be an indicator of a probiotic regimen or exogenous substance. The methods and devices of this application can be used to test for non-toxigenic C. difficile in a vegetative or spore state, which can indicate efficacy of a particular micro-biota exogenous substance as a probiotic. Similar techniques can be used to infer a general condition of a microbiota.

### Notes and Examples

The following, non-limiting examples, detail certain aspects of the present subject matter to solve the challenges and provide the benefits discussed herein, among others.

Example 1 is a method for predicting in vivo colonization and infection dynamics by quantifying bacterial spore germination in an ex vivo assay, the method comprising: creating an ex vivo assay including bacteria spores and a homogenized stool sample from an in vivo model; loading the ex vivo assay into a microfluidic chip; detecting vegetative bacteria and the bacteria spores by sampling the ex vivo assay in the microfluidic chip using impedance cytometry; and predicting colonization of the bacteria of the in vivo model based on the detected vegetative bacteria and bacteria spores.

In Example 2, the subject matter of Example 1 optionally includes wherein creating the ex vivo assay further includes: centrifuging a slurry to separate stool solids; and sterile-filtering supernatant from the slurry.

In Example 3, the subject matter of Example 2 optionally includes wherein creating the ex vivo assay further includes: co-culturing bacteria spores with the sterile-filtered supernatant.

In Example 4, the subject matter of Example 3 optionally includes wherein creating the ex vivo assay further includes: conditioning the bacteria spores in taurocholate or in fecal supernatant.

In Example 5, the subject matter of Example 4 optionally includes wherein creating the ex vivo assay further includes: germinating the bacteria spores in growth media.

In Example 6, the subject matter of any one or more of Examples 1-5 optionally include wherein the impedance cytometry is single-cell impedance cytometry.

In Example 7, the subject matter of Example 6 optionally includes wherein detecting vegetative bacteria includes: detecting an impedance phase for each cell sampled in the microfluidic chip; recording a number of events within an impedance phase range based on the detected impedance phase for each cell; and grouping the events based on an impedance phase of each event.

In Example 8, the subject matter of Example 7 optionally includes wherein predicting colonization of the bacteria of the in vivo model includes: recording a number of events within an impedance phase range based on the detected impedance phase for each cell; and grouping the events based on an impedance phase of each event.

In Example 9, the subject matter of Example 8 optionally includes wherein the impedance cytometry is conducted at 500 kHz, 2 MHz, and 10 MHz, and wherein a flowrate of cells through a sampling channel of the microfluidic chip is between 300 and 400 cells per second.

In Example 10, the subject matter of any one or more of Examples 1-9 optionally include wherein the bacteria spores comprise Clostridioides difficile and wherein the vegetative bacteria are Clostridioides difficile.

Example 11 is a method for quantifying bacterial spore germination, the method comprising: creating an ex vivo assay including bacteria spores and a homogenized stool sample; loading the ex vivo assay into a microfluidic chip; and detecting vegetative bacteria and the bacteria spores by sampling the ex vivo assay in the microfluidic chip using impedance cytometry.

In Example 12, the subject matter of Example 11 optionally includes wherein creating the ex vivo assay further includes: centrifuging a slurry to separate stool solids; and sterile-filtering supernatant from the slurry.

In Example 13, the subject matter of Example 12 optionally includes wherein creating the ex vivo assay further includes: co-culturing bacteria spores with the sterile-filtered supernatant.

In Example 14, the subject matter of Example 13 optionally includes wherein creating the ex vivo assay further includes: conditioning the bacteria spores in taurocholate or in fecal supernatant.

In Example 15, the subject matter of Example 14 optionally includes wherein creating the ex vivo assay further includes: germinating the bacteria spores in growth media.

In Example 16, the subject matter of any one or more of Examples 11-15 optionally include wherein the impedance cytometry is single-cell impedance cytometry.

In Example 17, the subject matter of Example 16 optionally includes wherein detecting vegetative bacteria includes: detecting an impedance phase for each cell sampled in the microfluidic chip; recording a number of events within an impedance phase range based on the detected impedance phase for each cell; and grouping the events based on an impedance phase of each event.

In Example 18, the subject matter of Example 17 optionally includes wherein the impedance cytometry is conducted at 500 kHz, 2 MHz, and 10 MHz.

In Example 19, the subject matter of any one or more of Examples 17-18 optionally include wherein a flowrate of cells through a sampling channel of the microfluidic chip is between 300 and 400 cells per second.

In Example 20, the subject matter of any one or more of Examples 11-19 optionally include wherein the bacteria spores comprise Clostridioides difficile and wherein the vegetative bacteria are Clostridioides difficile.

Example 21 is a method for quantifying bacterial spore germination, the method comprising: mixing bacteria spores with a homogenized stool sample; loading the mixture into a microfluidic chip; and detecting vegetative bacteria from the bacteria spores by sampling the mixture in the microfluidic chip using impedance cytometry.

In Example 22, the subject matter of Example 21 optionally includes wherein the impedance cytometry is single-cell impedance cytometry.

In Example 23, the subject matter of any one or more of Examples 21-22 optionally include conditioning the bacteria spores in taurocholate or in fecal supernatant before mixing the bacteria spores with the homogenized stool sample.

In Example 24, the subject matter of Example 23 optionally includes diluting the bacteria spores in growth media; and germinating the bacteria spores in the growth media before mixing the bacteria spores with the homogenized stool sample.

In Example 25, the subject matter of any one or more of Examples 21-24 optionally include μm wherein the microfluidic chip includes a detection channel having a depth of 30 μm and a width of 30 μm.

In Example 26, the subject matter of Example 25 optionally includes wherein a flowrate of cells through the channel is between 300 and 400 cells per second.

In Example 27, the subject matter of Example 26 optionally includes wherein the impedance cytometry is conducted at 500 kHz, 2 MHz, and 10 MHz.

In Example 28, the subject matter of Example 27 optionally includes wherein detecting vegetative bacteria by sampling the mixture includes: detecting an impedance phase for each cell; recording a number of events within an impedance phase range based on the detected impedance phase for each cell; and grouping the events based on an impedance phase of each event.

In Example 29, the subject matter of any one or more of Examples 21-28 optionally include wherein the bacteria spores are Clostridioides difficile and wherein the vegetative bacteria are Clostridioides difficile.

Example 30 is a non-transitory machine-readable medium including instructions, for quantifying bacterial spore germination, which when executed by a machine, cause the machine to: sample an ex vivo assay within a sampling channel of a microfluidic chip, the assay including bacteria spores and a homogenized stool sample; measure impedance phase for each cell within the sampling channel; detecting vegetative bacteria from the bacteria spores based on the measured impedance phase.

Example 31 is a method for quantifying bacterial spore germination by single-cell impedance cytometry for assessment of host microbiota susceptibility to infection as described herein.

In Example 32, the subject matter of Example 31 optionally includes each and every novel feature or combination of features disclosed herein.

Example 33 is a system for quantifying bacterial spore germination by single-cell impedance cytometry for assessment of host microbiota susceptibility to infection as described herein.

In Example 34, the subject matter of Example 33 optionally includes each and every novel feature or combination of features disclosed herein.

Example 35 is a computer-readable storage medium having computer-executable instructions stored thereon which, when executed by one or more processors, cause one or more computers to perform functions for quantifying bacterial spore germination by single-cell impedance cytometry for assessment of host microbiota susceptibility to infection as described herein.

In Example 36, the subject matter of Example 35 optionally includes each and every novel feature or combination of features disclosed herein.

In Example 37, the apparatuses or method of any one or any combination of Examples 1-36 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for predicting in vivo colonization and infection dynamics by quantifying bacterial spore germination in an ex vivo assay, the method comprising:
creating an ex vivo assay including bacteria spores and a homogenized stool sample from an in vivo model;
loading the ex vivo assay into a microfluidic chip;
detecting vegetative bacteria and the bacteria spores by sampling the ex vivo assay in the microfluidic chip using impedance cytometry; and
predicting colonization of the bacteria of the in vivo model based on the detected vegetative bacteria and bacteria spores.

2. The method of claim 1, wherein creating the ex vivo assay further includes:
centrifuging a slurry to separate stool solids; and
sterile-filtering supernatant from the slurry.

3. The method of claim 2, wherein creating the ex vivo assay further includes:
co-culturing bacteria spores with the sterile-filtered supernatant.

4. The method of claim 3, wherein creating the ex vivo assay further includes:
conditioning the bacteria spores in taurocholate or in fecal supernatant.

5. The method of claim 4, wherein creating the ex vivo assay further includes:
germinating the bacteria spores in growth media.

6. The method of claim 1, wherein the impedance cytometry is single-cell impedance cytometry.

7. The method of claim 6, wherein detecting vegetative bacteria includes:
detecting an impedance phase for each cell sampled in the microfluidic chip;
recording a number of events within an impedance phase range based on the detected impedance phase for each cell; and
grouping the events based on an impedance phase of each event.

8. The method of claim 7, wherein predicting colonization of the bacteria of the in vivo model includes:
recording a number of events within an impedance phase range based on the detected impedance phase for each cell; and
grouping the events based on an impedance phase of each event.

9. The method of claim 8, wherein the impedance cytometry is conducted at 500 kHz, 2 MHz, and 10 MHz, and wherein a flowrate of cells through a sampling channel of the microfluidic chip is between 300 and 400 cells per second.

10. The method of claim 1, wherein the bacteria spores comprise Clostridioides difficile and wherein the vegetative bacteria are Clostridioides difficile.

11. A method for quantifying bacterial spore germination, the method comprising:
creating an ex vivo assay including bacteria spores and a homogenized stool sample;
loading the ex vivo assay into a microfluidic chip; and
detecting vegetative bacteria and the bacteria spores by sampling the ex vivo assay in the microfluidic chip using impedance cytometry.

12. The method of claim 11, wherein creating the ex vivo assay further includes:
centrifuging a slurry to separate stool solids; and
sterile-filtering supernatant from the slurry.

13. The method of claim 12, wherein creating the ex vivo assay further includes:
co-culturing bacteria spores with the sterile-filtered supernatant.

14. The method of claim 13, wherein creating the ex vivo assay further includes:
conditioning the bacteria spores in taurocholate or in fecal supernatant.

15. The method of claim 14, wherein creating the ex vivo assay further includes:
germinating the bacteria spores in growth media.

16. The method of claim 11, wherein the impedance cytometry is single-cell impedance cytometry.

17. The method of claim 16, wherein detecting vegetative bacteria includes:
detecting an impedance phase for each cell sampled in the microfluidic chip;
recording a number of events within an impedance phase range based on the detected impedance phase for each cell; and
grouping the events based on an impedance phase of each event.

18. The method of claim 17, wherein the impedance cytometry is conducted at 500 kHz, 2 MHz, and 10 MHz.

19. The method of claim 17, wherein a flowrate of cells through a sampling channel of the microfluidic chip is between 300 and 400 cells per second.

20. The method of claim 11, wherein the bacteria spores comprise Clostridioides difficile and wherein the vegetative bacteria are Clostridioides difficile.

21-30. (canceled)
